(19) United States
(12) Patent Application Publication
(10) Pub. No.: US 2008/0208144 A1
(43) Pub. Date: Aug. 28, 2008

(54) Syringe Device and a Method of Assembling the Same

(75) Inventor: Claus Schmidt Moller, Fredensborg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/995,591

(22) PCT Filed: Jul. 17, 2006
(86) PCT No.: PCT/EP06/07002

§ 371 Date: Feb. 14, 2008

(30) Foreign Application Priority Data
Jul. 25, 2005 (EP) 05016075.3

(51) Int. Cl. A61M 5/315

(52) U.S. Cl. 604/220

(57) ABSTRACT

The present invention relates to a method of assembling a syringe device such that rotational and translational tolerances are reduced or eliminated. The syringe device comprises a rotational retaining mechanism arranged to prevent backwards movement of a piston rod. The method comprises the step rotating the rotational retaining mechanism into a locking position prior to completing the assembly. Furthermore, the invention relates to a syringe device comprising a retaining mechanism having a first part and a second part, said parts being interconnected by a plurality of elements, wherein one of said elements comprises a first part and a second part separated by an interconnecting seam.

### FIELD OF THE INVENTION

The present invention relates to a method of assembling a syringe device comprising a medicament to be ejected. In particular, the present invention relates to a method of assembling a syringe device comprising a plurality of elements rotatable in relation to each other. Furthermore, the present invention relates to a syringe device obtained by the method according to the invention.

### BACKGROUND OF THE INVENTION

In connection with use of syringe devices for ejecting a medicament into the body of a human being, a known problem is that blood may be sucked into the syringe device. This is undesirable as the blood may coagulate inside the pen and block the needle. With the increased focus on miniaturisation of components for syringe devices, the cross-sectional area of the flow channel of needles is decreasing, and, thus, coagulated elements may block the needle more easily. Accordingly, the need for syringe devices avoiding or limiting the risk of blood in the pen is also increased.

Furthermore, as the suction of blood into the syringe device may be caused by translational and rotational tolerances between elements of the syringe device there is a need for reducing or even eliminating the tolerances between components. Normally, the tolerances are reduced by manufacturing the components on high-precision machines using high-precision tools. However, such machines and tools are expensive, and, thus, the manufacturing cost is increased.

It is an object of a preferred embodiment of the present invention to provide a method of manufacturing a syringe device wherein tolerances in the assembled syringe device is reduced or eliminated without using high-precision manufacturing devices.

### SUMMARY OF THE INVENTION

In order to overcome the above problem the present invention relates in a FIRST aspect to a method of assembling a syringe device comprising:
an ejection mechanism for ejecting a dose of a medicament from the syringe device, the ejection mechanism comprising:
   a housing;
   a piston rod for ejecting the medicament from the syringe device by forward movement of the piston rod relative to the housing;
   a driver for driving the piston during ejection of the medicament from the syringe device, the driver being rotatable relative to the housing; and
   a retaining mechanism arranged to prevent backwards movement of the piston rod, the retaining mechanism comprising a first retaining member engaging a second retaining member, the first and second retaining members being unidirectionally rotatable relative to each other in a predetermined rotational direction, the first and the second retaining members furthermore being interconnected via an interconnection assembly comprising a plurality of elements, the plurality of elements including the driver and at least one further element;
      the method comprising steps of:
providing the interconnection assembly except from one of said plurality of elements; and subsequently
assembling the first and second retaining members by positioning said retaining members in a locking position, in which the retaining members are locked against rotation in a direction opposite to said predetermined rotational direction; and subsequently
completing the interconnection assembly.

One advantage of the present invention is that the tolerances are substantially eliminated by completing the assembly after the first and the second retaining members are positioned in the locking position. Accordingly, when the syringe device is in its initial position, i.e. set on a dose corresponding to zero international units (IU), the piston rod cannot be moved backwards, and, thus, blood cannot be sucked into the syringe device.

A further advantage of the present invention is that precision of the dose setting mechanism is increased as the rotational and translational tolerances are eliminated or even reduced. Accordingly, when a dose is set to a specific dose e.g. such that a dose indicating scale indicates a set dose of 38 IU, the risk of the dose setting mechanism being set to a different dose, such as 37 IU or 39 IU, is reduced or eliminated.

In one embodiment the step of providing the interconnecting assembly is carried out prior to the step of assembling the first and second retaining members, which is carried out prior to the step of completing.

In one embodiment the piston rod has a threaded outer surface for engagement with threaded inner surface of the housing. Accordingly, relative rotational movement between the piston rod and the housing results in translational movement of the piston rod. In the alternative the piston rod and the housing are locked for relative rotational movement e.g. by provision of a groove and tongue arrangement.

The driver and the piston rod may be locked for relative rotational movement while allowing relative translational movement. In the latter case the piston rod may change position when the driver is rotated, while the driver remains essentially in the same translational position. The driver may be connected to a dose setting member and a button part of the syringe device in such a way that during dose setting the driver remains in the same translational and rotational position, whereas the driver rotates and causes the piston rod to rotate, when the button in pushed in the direction of the needle.

The first and the second retaining member are unidirectional rotatable between a plurality of predetermined locking positions. The number of locking positions may correspond to at least the number of increments and/or IU ejected when the piston rod is rotated one revolution.

In one embodiment the unidirectional lock is changeable between a predetermined number of positions. The predetermined number of positions may be defined by a corresponding predetermined number of indentations e.g. provided on an inner surface of the second retaining member and being adapted to be engaged by engaging arms of the first retaining member. Alternatively, the arms are provided on the second member, while the indentations are provided on the first member.

In another embodiment the unidirectional lock is changeable between an infinite number of positions. This may be achieved by adapting one of the retaining members to slide on a surface of the other retaining member and by providing a high coefficient of friction between the sliding surfaces.

In the latter embodiment an inner surface of the second retaining member may be smooth/even while the first retaining member comprises arms adapted to slide on said surface and at least one of the engaging surfaces may comprise a rubber like material having a high frictional coefficient. Naturally, it will be appreciated that said engaging arms may be provided on the second retaining member while the smooth surface may be provided on the first retaining member. Furthermore, it will be appreciated that the surfaces of the abovementioned indentations and the corresponding engaging arms may have a high coefficient of friction.

The first and second retaining members are interconnected via two paths. A first path defined by the aforementioned engagement between arms and indentations, and/or arms and surfaces with a high frictional coefficient. A second path is defined by the interconnection assembly. The interconnecting assembly comprises a plurality of elements including the driver and at least one further element. Said further element may be the dose setting member and/or the housing and/or a cylindrical element used to indicate the set dose.

In the context of the present invention the term backwards movement shall be understood as movement in a direction opposite the direction the piston rod moves when a dose is ejected, i.e. towards the button and away from the needle part of the syringe device.

In the context of the present invention the term initial position is the position the syringe device is positioned in prior to setting a dose and after the dose has been injected, i.e. where a needle of the syringe device may still penetrate the skin of a patient, and, thus, a situation wherein any backwards movement of the piston rod may cause blood to be sucked into the syringe device.

In order to eliminate or reduce the accumulated tolerances (i.e. tolerances accumulated from one element to the other in a string of mechanically interconnected elements) in the interconnecting assembly, and, thus, to ensure that the piston rod may not move backwards when the device is in the initial position, the elements of the device are assembled in a predetermined order of steps:

The first step comprises: providing the interconnection assembly except from one of said plurality of elements, thus, allowing the first and the second retaining members to rotate relative to each other in the second step.

The second step comprises: assembling the first and second retaining members by positioning said retaining members in a locking position, in which the retaining members are locked against rotation in a direction opposite to said predetermined rotational direction. This locking position may be achieved by assembling the first and the second retaining members and rotating the two members in a direction opposite the unidirectional lock until relative rotation in said opposite direction is no longer possible. In embodiments wherein one of the retaining members comprises indentations while the other retaining member comprises arms for engaging said indentations, the arms may initially not be positioned at a button part of the indentation, and, thus, not be positioned in the locking position. In the latter embodiment, rotation in said opposite direction ensures that the retaining mechanism is moved into the locking position prior to the third step.

The third step comprises: completing the interconnection assembly. This third step may comprise the steps of providing the excepted element and securing said element to another element in the plurality of elements.

The step of securing (in the abovementioned third step) may comprise the step of securing the excepted element to another element in the plurality of elements by means of an adhesive, e.g. by providing an adhesive and curing the adhesive. The adhesive may be provided on a surface of the excepted element or a surface to which the excepted element is to be attached. The step of curing may comprise the step of applying heat to the adhesive and/or irradiating the adhesive, e.g. with a UV-source, so as to accelerate the curing process.

Alternatively, or as a supplement, the step of securing may comprise the step of securing the excepted element to another element in the plurality of elements by means of laser welding.

The step of providing may include a step of rotating the excepted element relative to another element in the plurality of elements so as to reduce the rotational and/or the translational tolerances in the interconnecting assembly.

According to a SECOND aspect the present invention relates to a syringe device comprising an ejection mechanism for ejecting a dose of a medicament from the syringe device, the ejection mechanism comprising:
a housing;
a piston rod for ejecting the medicament from the syringe device by forward movement of the piston rod relative to the housing;
a driver for driving the piston during ejection of the medicament from the syringe device, the driver being rotatable relative to the housing; and
a retaining mechanism arranged to prevent backwards movement of the piston rod, the retaining mechanism comprising a first retaining member engaging a second retaining member, the first and second retaining members being unidirectionally rotatable relative to each other in a predetermined rotational direction, the first and the second retaining members furthermore being interconnected via an interconnection assembly comprising a plurality of elements, the plurality of elements including the driver and at least one further element,
   wherein one of the elements in the plurality of elements comprises a first part and a second part separated by an interconnecting seam and wherein the first and second retaining members are interconnected via said interconnecting seam.

An advantage of the interconnecting seam is that the first and second retaining members may be positioned in a locking position prior to attaching the first part and the second to each other. Accordingly, the accumulated tolerances may be reduced or even eliminated as described under the first aspect of the invention.

The seam may be a welding seam provided by means of laser welding. In order to facilitate laser welding an absorption coefficient of the first part at a predetermined optical wavelength may be higher than an absorption coefficient of the second part at the wavelength, or vice versa. Accordingly, the laser beam may be directed through the poorly absorbing part and whereby the transition between the first and the second part may be heated so as to accomplish the laser welding.

Alternatively, or as a supplement at least a part of the seam may be a welding seam provided by means of ultrasonic welding.

In the context of the present invention a seam also comprises a cured adhesive, and, thus, in one embodiment the seam comprises such a cured adhesive. The adhesive may be a two-component adhesive such as Cyanoarylat.

The invention according to the second aspect of the invention may comprise any feature and/or element of the first aspect of the invention, and vice versa. As an example, any definition of the first aspect of the invention also applies to the second aspect of the invention and vice versa.

### DESCRIPTION OF THE DRAWINGS

The invention will now be described in further detail with reference to the drawings, in which:

FIG. 1 discloses an isometric drawing of a first embodiment of the invention,

FIG. 2 discloses section A-A of FIG. 1,

FIG. 3 discloses the second retaining member of FIGS. 1 and 2, and

FIG. 4 discloses a second embodiment of the invention.

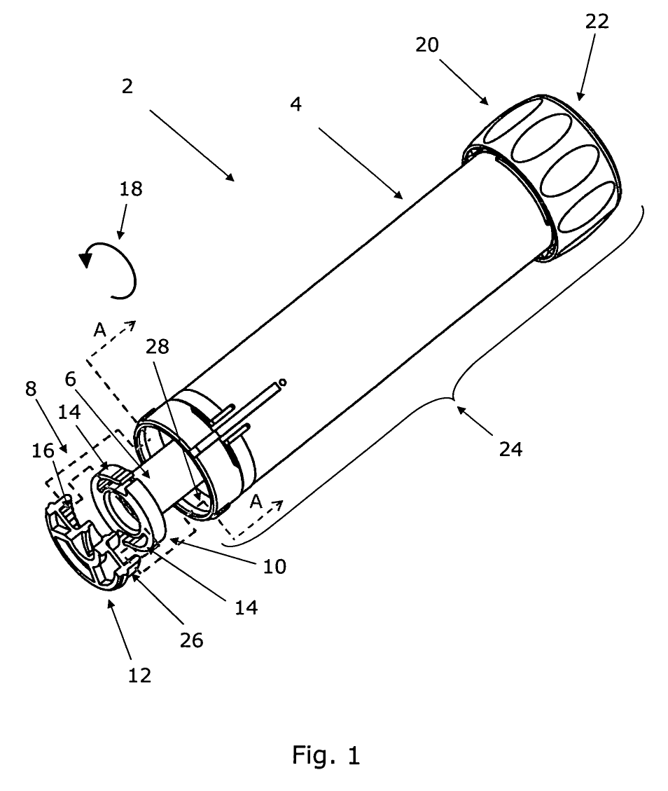

FIG. 1 discloses a syringe device having a lower part (not shown) and an upper part 2. The upper part comprises a housing 4, a piston rod (not shown), a driver 6 and a retaining mechanism 8 having a first retaining member 10 and a second retaining member 12. The first retaining member comprises two engaging arms 14 adapted to engage indentations 16 provided on the inner surface of the second retaining member 12. Due to the engagement between the arms 14 and the indentations 16 the driver may only rotate in the direction indicated by arrow 18 when the second retaining member is attached to the housing 4.

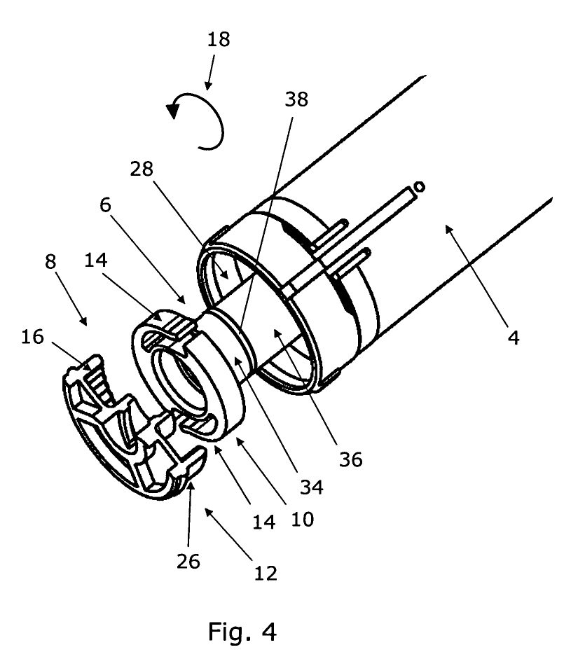

When the syringe device is completely assembled (not shown in FIG. 1) the first and second retaining members are interconnected not only via the indentations 16 and the arms 14 but also via an interconnecting assembly defined by at least the housing 4 (to which the second retaining member is attached when the device is assembled) and the driver 6. Accordingly, it will be appreciated that the housing 4 and the driver 6 are coupled to each other when the device is assembled, this coupling may be provided by means of a plurality of elements such as disclosed in FIG. 4.

The device further comprises a dose setting member 20 comprising a button 22. In one embodiment a dose is set by rotating the dose setting member in the direction opposite arrow 18. During said dose setting, the driver 6 remains in the same rotational position, relative to the housing 4. When the dose has been set, the medicament (not shown) may be ejected by pressing the button 22 in the distal direction, i.e. towards a needle portion (not shown) provided in the first part and in the opposite end of the device as the button 22. When the button 22 is pressed the driver 6 rotates in the direction indicated by arrow 18 whereby the piston rod (not shown) is forced to rotate in the same direction.

In the embodiment of FIG. 1 the syringe device is assembled in the following order. In a first step a semi-assembled part indicated by brace 24 is assembled. The semi-assembled part comprises the housing 4, the driver 6 and the interconnecting assembly described above.

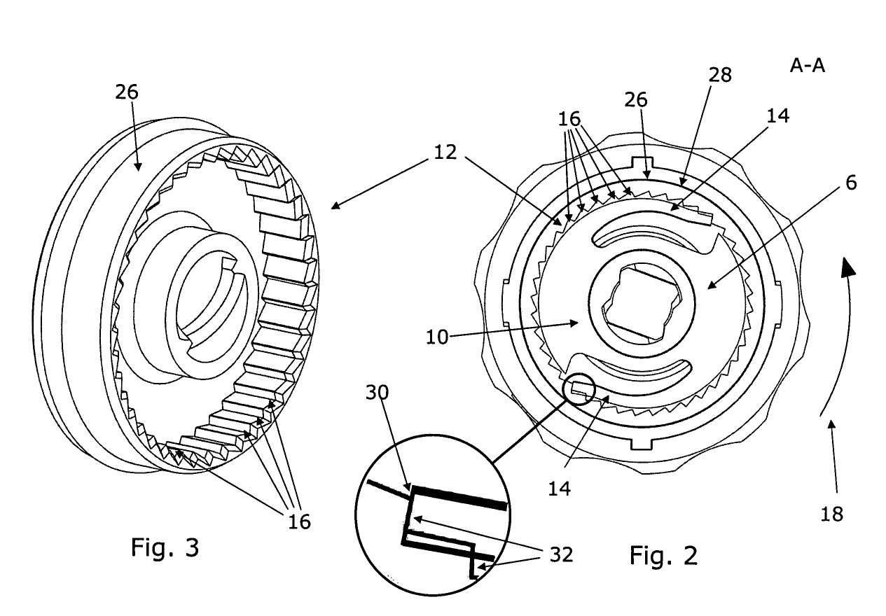

In a second step the first retaining member 10 and the second retaining member 12 are position in relation to each other such that the engaging arms engage an indentation and such that no relative rotational movement between said two elements, in a direction opposite the direction indicated by arrow 18, is possible (this is described in further detail below in relation to FIG. 2). In order to ensure that the second retaining member is positioned correctly in relation to the first retaining member, the second retaining member may be forced in the direction indicated by arrow 18.

In a third step the outer surface 26 of second retaining member 12 is attached to the inner surface 28 of the housing 4. The attachment may be established by means of an adhesive (e.g. curable by means of heat or radiation), welding or any other attachment method providing a seam between the second retaining member 12 and the housing 4.

FIG. 2. discloses the cross-section A-A indicated in FIG. 1. In FIG. 2 the two engaging arms 14 of the first retaining member 10 engage indentations 16 of the second retaining member 12. Naturally it will be appreciated that in different embodiments there may be provided fewer or further engaging arms, such as one, three, four, five or six arms. Analogously, the number of indentations 16 may be varied. In one embodiment the number of indentations corresponds to the number of IU expelled from the device when the piston rod has performed one revolution.

In the second step described above the first retaining member 10 and the second retaining member 12 is being position in relation to each other, such that the engaging arms 14 engage an indentation 16. This is done by rotating the second retaining member 12 in the direction opposite the direction indicated by arrow 18. By engaging an indentation is meant, that an end surface 30 of an arm 14 engages an indentation surface 32 in such a way that no relative rotation, between the first and the second retaining members, is possible in the direction opposite the direction indicated by arrow 18. It will be appreciated that the lower angle between the axial direction of the device and the end surface 30 and/or the indentation surface is, the more efficient is the rotational lock.

The advantage of the second step is that any rotational tolerance in the interconnecting assembly is eliminated, as the second retaining member 12 is attached to the housing after the second step. It will be appreciated that translational tolerances transforms into a rotational tolerance for elements which are designed to move in the translational direction when rotated (i.e. elements comprising threaded engagement with another element), and, thus, such translational tolerances are also eliminated. The advantage is that when the set dose corresponds to zero IU the driver (and thus the piston rod) cannot rotate in the direction opposite the direction indicated by arrow 18, and, thus, the piston cannot be drawn in the proximal direction. The consequence is that blood cannot be sucked into the syringe device.

FIG. 3 illustrates the second retaining member comprising a plurality of indentations 16.

FIG. 4 illustrates a second embodiment of the invention wherein the driver 6 comprises a first part 34 and a second part 36 separated by an air gab 38. In the embodiment of FIG. 2 the syringe device is assembled in the following order.

In a first step the housing, the second part 36 and the remaining interconnecting member(s) are assembled.

In the second step, the first part 34 which comprises the engaging arms 14 inserted into the second retaining member 12 such that the arms engage an indentation, as described above. The second retaining member is then attached to the housing, such that the outer surface 26 of second retaining member 12 is attached to the inner surface 28 of the housing 4.

In a third step the first part 34 and the second part 36 are attached to each other, e.g. by means of laser welding. If the two parts are attached to each other by means of laser welding the laser beam may be directed through the first part 34, which may have a lower coefficient of absorption than the second part 36. In the alternative, the second step may comprise applying an adhesive to en end surface of the first part 34 prior to moving the second part into a position wherein it engages the housing. In said alternative the third step may comprise the step of applying external radiation or heat to the syringe device so as to accelerate the curing process.

1. A method of assembling a syringe device comprising:
an ejection mechanism for ejecting a dose of a medicament from the syringe device, the ejection mechanism comprising:
a housing;
a piston rod for ejecting the medicament from the syringe device by forward movement of the piston rod relative to the housing;
a driver for driving the piston during ejection of the medicament from the syringe device, the driver being rotatable relative to the housing; and
a retaining mechanism arranged to prevent backwards movement of the piston rod, the retaining mechanism comprising a first retaining member engaging a second retaining member, the first and second retaining members being unidirectionally rotatable relative to each other in a predetermined rotational direction, the first and the second retaining members furthermore being interconnected via an interconnection assembly comprising a plurality of elements, the plurality of elements including the driver and at least one further element;
the method comprising steps of:
providing the interconnection assembly except from one of said plurality of elements; and subsequently
assembling the first and second retaining members by positioning said retaining members in a locking position, in which the retaining members are locked against rotation in a direction opposite to said predetermined rotational direction; and subsequently
completing the interconnection assembly.

2. A method according to claim 1, wherein the step of completing comprises the steps of providing the excepted element and securing said element to another element in the plurality of elements.

3. A method according to claim 1, wherein the step of securing comprises the step of securing the excepted element to another element in the plurality of elements by means of an adhesive.

4. A method according to claim 3, wherein the step of securing comprises the steps of providing an adhesive and curing the adhesive.

5. A method according to claim 1, wherein the step of securing comprises the step of securing the excepted element to another element in the plurality of elements by means of laser welding.

6. A method according to claim 1, wherein the step of providing includes the step of rotating the excepted element relative to another element in the plurality of elements so as to reduce the rotational and/or the translational tolerances in the interconnecting assembly.

7. A syringe device comprising an ejection mechanism for ejecting a dose of a medicament from the syringe device, the ejection mechanism comprising:
a housing;
a piston rod for ejecting the medicament from the syringe device by forward movement of the piston rod relative to the housing;
a driver for driving the piston during ejection of the medicament from the syringe device, the driver being rotatable relative to the housing; and
a retaining mechanism arranged to prevent backwards movement of the piston rod, the retaining mechanism comprising a first retaining member engaging a second retaining member, the first and second retaining members being unidirectionally rotatable relative to each other in a predetermined rotational direction, the first and the second retaining members furthermore being interconnected via an interconnection assembly comprising a plurality of elements, the plurality of elements including the driver and at least one further element;
wherein one of the elements in the plurality of elements comprises a first part and a second part separated by an interconnecting seam and wherein the first and second retaining members are interconnected via said interconnecting seam.

8. A syringe device according to claim 7, wherein the seam is a welding seam provided by means of laser welding.

9. A syringe device according to claim 8, wherein an absorption coefficient of the first part at a predetermined optical wavelength is higher than an absorption coefficient of the second part at the wavelength.

10. A syringe device according to claim 7, wherein the seam is a welding seam provided by means of ultrasonic welding.

11. A syringe device according to claim 7 wherein the seam comprises a cured adhesive.
